(19) United States
(12) Patent Application Publication
(10) Pub. No.: US 2026/0251630 A1
(43) Pub. Date: Aug. 27, 2026

(54) INTELLIGENT AGENTS AND SYSTEMS FOR CONDITION SENSITIVE ITEMS

(71) Applicant: Paul Atkinson, Poway, CA (US)

(72) Inventor: Paul Atkinson, Poway, CA (US)

(21) Appl. No.: 19/060,469

(22) Filed: Feb. 21, 2025

(51) Int. Cl. G01N 33/15; G16H 20/10

(52) U.S. Cl. CPC G01N 33/15; G16H 20/10

(57) ABSTRACT

Briefly, an intelligent agent for physical items is disclosed that operates autonomously, and over time progressively transforms sensed conditions into measures of the item's utility, evaluates the measures, makes decisions and performs actions, according to predetermined conditions.

### RELATED APPLICATIONS

This application claims priority to U.S. provisional application 63/556,465 filed Feb. 22, 2024, and entitled “Intelligent Agents and Systems for Temperature Sensitive Items”. This application also claims priority to U.S. provisional application 63/749,060 filed Jan. 25, 2025, and entitled “Progressive Agency”; U.S. provisional application 63/720,928 filed Nov. 15, 2024 and entitled “Replacement Prescription Systems & Methods”; U.S. provisional application 63/706,106 filed Oct. 11, 2024 and entitled “Digital Health Platforms with Medication Agents”; U.S. application Ser. No. 18/742,852 filed Jun. 13, 2024, and entitled “Ad hoc Power Enabled Sensing Devices”; and U.S. patent application Ser. No. 18/610,508 filed Mar. 20, 2024 “Ad hoc Power Enabled Fail-Safe Systems”.

This application also relates to:
U.S. application Ser. No. 17/890,131 filed Aug. 17, 2022 and U.S. provisional applications 63/389,939 filed Jul. 17, 2022, 63/357,730 filed Jul. 1, 2022 and 63/247,489 filed Sep. 23, 2021, each entitled “Ad Hoc Power Enabled Communication”; and U.S. provisional application 63/233,916 filed Aug. 17, 2021 and entitled “Ad hoc Communication”;
U.S. provisional application 63/453,316 filed Mar. 20, 2023, and entitled Ad hoc Power Enabled Fail-Safe Systems;
U.S. application Ser. No. 18/431,999 filed Feb. 4, 2024, and U.S. provisional applications 63/472,645 filed Jun. 13, 2023, 63/448,305 filed Feb. 26, 2023, and 63/443,510 filed Feb. 6, 2023, each entitled Ad Hoc Power Enabled Sensing Devices;
U.S. application Ser. No. 18/213,770 filed Jun. 23, 2023, and entitled Hybrid Wireless Communications; 63/392,770 filed Jul. 27, 2022, and 63/354,757, filed Jun. 23, 2022, both entitled Hybrid Wireless Smart Tags;
2, 2024, U.S. Pat. No. 10,853,902 granted Dec. 1, 2020, each entitled “Agents and Systems for Rights Management”;
U.S. application Ser. No. 18/199,178 filed Mar. 8, 2023 and U.S. provisional application 63/439,312 filed Jan. 17, 2023, both entitled “Autonomous Agents Using Smart Contracts To Enable Pay-For-Performance Transactions”; U.S. provisional applications 63/436,227 filed Dec. 30, 2022 and U.S. provisional applications 63/338,962 filed May 6, 2022, both entitled Agents and Systems for Rights Management, and U.S. provisional application 63/317,639 filed Mar. 8, 2022 and entitled CHR-048-CXP.
U.S. Pat. No. 11,394,547 filed Aug. 16, 2016, U.S. patent application Ser. No. 17/734,364 filed May 2, 2022 and U.S. provisional applications 62/354,491 filed Jun. 24, 2016 and 62/358,685, each entitled “Transaction Agents and Systems”.

Each of the patents, applications and provisional applications listed above, and elsewhere herein, are included herein by reference.

### FIELD OF THE INVENTION

The field of invention includes novel item-level, autonomous computing devices—intelligent agents—and related systems and methods. Over the lifetime of an item (e.g., a medication), an intelligent agent progressively senses one or more conditions to which the item is sensitive, transforms the sensed conditions into measures of the item's utility (e.g., a medication's fitness-for-use), evaluates the measures, makes decisions and performs actions according to predetermined conditions.

### BACKGROUND OF THE INVENTION

The healthcare of hundreds of millions of people depends on the efficacy of temperature sensitive medications. Yet today, there is no way of knowing if the individual doses of the medications they take are fit-for-use when they take them: that their efficacy hadn't been substantively degraded because of their lifetime exposure to temperature. The negative consequences (which are largely unattributed to loss of efficacy due to exposure to temperature) ripple across the stakeholders in medications and their outcomes: patients, families, caregivers, healthcare providers, pharmaceutical companies, distributors, pharmacies, payers, employers etc.

What's needed are intelligent agents for individual pill bottles and pill packs, vials and syringes, inhalers, injectors, fluid bags etc. that autonomously and progressively determine the fitness-for-use—the utility—of the medications stored within them. And it's needed for many other items, goods and things (collectively referred to herein as “items”) as well.

### SUMMARY OF THE PRESENT INVENTION

An intelligent agent (“IA”) is an item-level computing device configured to be affixed or bound to, embedded in or integrated with, an item or its packaging or container, such that the stakeholders in the item (and its use and outcomes) can be confident it will remain physically associated with the item over it's useful life. Once activated an IA operates autonomously and progressively according to predetermined conditions, and executes processes such as:
Sensing, perceiving one or more conditions such as ambient temperature;
Determining the cumulative exposure to temperature (“CET”) of an item over its lifetime;
Converting the determined CET into a determination of fitness-for-use, and the determined fitness-for-use into a one or more measures that can be easily communicated to, understood and trusted by, local and remote, custodial and noncustodial, “stakeholders” in the medication and its outcomes (e.g., patients and caregivers, clinicians, healthcare providers, pharmaceutical companies, payers, etc.);
Evaluating the measures of fitness-for-use, make decision(s) based on the results of the evaluations, and accordingly;
Performing actions such as:
   informing, alerting, messaging to or communicating with, patients and other stakeholders (local and remote);
   executing transactions with local and remote devices and systems
   recording/storing (and advantageously generating proofs of) determinations and measures of CET and fitness-for-use and their determinants, dependents and derivatives, actions taken/performed, and transactions executed by an IA, so that the IA (and by extension patients and/or other stakeholders/systems in receipt of such information) can for example:
      authorize replacements of expired, or soon-to-be expired medications
      increase compliance with storage and handling protocols, and patient adherence to prescriptions
      reduce a significant barrier to direct-to-patient healthcare
      improve inventory management, reduce waste (predictive expiration, useful life—and allocation/distribution/consumption prioritized accordingly)
      distribute costs and benefits of among stakeholders, pay for performance
      improve and accelerate the development of medications and related therapies.

### Terminology

Medications are the exemplar used to describe and illustrate the inventions disclosed herein which, as noted elsewhere, are applicable to a wide range of other items. As the terms are used herein, “fitness-for-use”, “efficacy”, “viability” and more generally “utility”, are the power, capacity or ability of a medication (or item) to perform an intended function, achieve a goal or purpose, its usefulness. An item's fitness-for-use, it's utility, may depend on the condition or state, absence or presence of, one or more substances in the item. Such substances in medications are referred to as “therapeutic agents”. Depending on the context the term ‘medication’ may refer to a ‘therapeutic agent.’

As the term is used herein, a “determination” is the result or outcome of a process, typically a value, metric or measure. And “shelf life” is the anticipated length of time an item (e.g., a medication) stored under specific conditions (across all locations) will remain fit-for-use, and “expiration date” is a predetermined date that anticipates when an item will no longer be fit-for-use. Using determinations or measures of CET or fitness-for-use etc., an item's shelf life and expiration date may be predicted by an IA (e.g., according to predetermined conditions, current conditions, trends etc.). And according to predetermined conditions, it's expiration date may be progressively revised by an IA in response to sensed or perceived conditions or their derivatives and dependents (e.g., determinations of CET, measures of fitness-for-use); or subsequent evaluations and decisions based on them.

As used herein the term “useful life” refers to the actual length of time an item is fit-for-use. And the terms “life cycle” and “lifetime” as used herein are the same: the period of time from manufacture (or earliest association with an IA) until the item is no longer fit-for-use. Note that an IA may continue to operate in some capacity beyond the useful life of an item to which it is physically associated, e.g., to facilitate a return, trade-in, exchange, or replacement, or disposal or recycling etc. and enable related actions and transactions (issuance of rebates, credits, release of security interests etc.).

As the terms are used herein, “measure” and “metric” are substantially similar and generally substitutable for each other. Both are used for quantitative comparison of properties, attributes, with measure used for more concrete or objective attributes and metric for more abstract, higher-level, or somewhat subjective attributes. Similarly, the terms “activation”, “initiation” and “trigger” are substantially similar and generally substitutable for each other although there are circumstances where the meaning may be substantively different: “activate” means to start to or enable a device or circuitry, or process; “initiate” means to start, to begin a process and “trigger” means to initiate (or activate) an action or process in response to an event or other stimulus, e.g., an ambient condition, a pulse from an electronic circuit, a measure generated by a computational process. The terms “determine”, “compute” and “calculate” are substantially similar and generally substitutable for each other although there are circumstances where the meaning may be substantively different. The term ‘determine’ is the broadest of the three, and encompasses the other two, and as used herein refers to an act or process for producing a result (e.g., a measure or metric, by way of calculation, computation and/or other means, methods, processes).

The term “conditions” (vs. “predetermined conditions”) refers to those of an item, its ambient environment, package or container or other item in the ambient environment, or an associated IA etc. The term “cumulative condition” refers to progressive determinations of cumulative sensed conditions over time. CET is an example of a cumulative condition (temperature). Conditions are generally those that can be sensed or perceived, while predetermined conditions are akin to criteria that are embedded or stored in, the control circuitry of an IA, and according to which, in whole or in part, the IA autonomously executes processes and performs actions.

### BRIEF DESCRIPTION OF THE DRAWINGS

FIG. 1 illustrates an exemplary IA subsystem.

FIG. 2 illustrates an exemplary IA configured to operate according to predetermined conditions, and accordingly perform actions; execute processes once activated.

FIG. 3 illustrates by way of dotted line 225-A, how processes in FIG. 2 may vary in response to events and processes and their outcomes/results.

FIG. 4 illustrates by way of a dotted line 227-A, how processes in FIG. 2 may vary in response to events and processes and their outcomes/results.

FIG. 5 illustrates how a computed MKT is both a determination of CET and a determination of fitness-for-use (Fitness-for-Use).

FIG. 6 illustrates exemplary processes from sensing to performing actions.

FIG. 7 is a flow diagram of an exemplary implementation of an IA system for providing a temperature-based viability assessment of a therapeutic agent.

FIG. 8 is a flow diagram of a computational process of a basic temperature based MKT system.

FIG. 9 illustrates an exemplary computational process, data transformation.

FIG. 10 illustrates an exemplary computational process, data transformation.

FIG. 11 is a data flow diagram associated with the calculations found in FIG. 7.

FIGS. 12a, 12b, 12c and 12d illustrate flow processes for a first scenario.

FIGS. 13a, 13b, 13c and 13d illustrate flow processes for a second scenario.

FIGS. 14a, 14b, 14c and 14d illustrate flow processes for a third scenario.

### DETAILED DESCRIPTION

### Intelligent Agents (IA's)

Fit-for-purpose, item-level IA's may be single-use or multi-use, flexible or rigid, embedded in or integrated with, or otherwise physically associated with an item as previously described. IA's may also be configured for use with multi-item cartons, boxes, and other mobile or transportable containers.

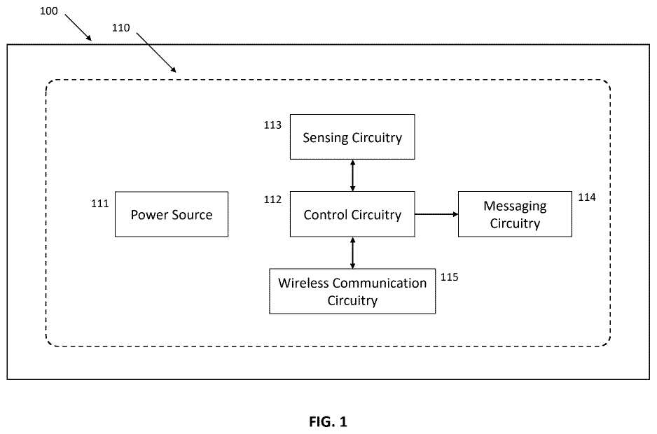

An exemplary IA 100 is illustrated in FIG. 1 that comprises an embedded system 110, the embedded system comprising:
A power source 111
   preferably an onboard battery and/or an energy harvester
   and/or an interface to an external power source that is part of, or physically associated with, the item or the IA
Control Circuitry 112 comprising:
   a controller, logic, memory
   primitives, functions (hardware, firmware)
   operating instructions, programs, systems
   predetermined conditions
   a clock/timer
Sensing Circuitry 113 configured fit-for-purpose to sense one or more ambient conditions (or those of the item), or more generally to “perceive” the item's environment or the item itself (e.g., it's condition, state or status).
Messaging Circuitry 114 (e.g., visual (reflective, transreflective), optical (emissive), audible, tactile) for messaging alerts, notices and in general, data or information; outbound, unidirectional.
Communication Circuitry 115 (e.g., EPCgen2/RAIN RFID, NFC, Bluetooth/BLE, Thread, WIFI, cellular, satellite, wired) may be configured for example:
   as a beacon or transmitter for example, to broadcast an identifier or “advertisement” for “discovery” by appropriately enabled devices such as mobile phones, tablets, notebooks or computers, or networks, or
   to respond to a wireless signal e.g., such as a query for or to update, the state, status or condition data or information (the query in response to, or regarding data or information related to messaging by messaging circuitry, e.g., a visual alert that prompts the query; receiving location data (e.g., GPS).
   Passive (batteryless), active or ad hoc power enabled as disclosed in U.S. patent application Ser. No. 17/890,131 entitled Ad hoc Power Enabled Communication.
In some instances, the communication circuitry may include ‘wired’ communication circuitry, e.g., via surface contacts or connectors for ad hoc communications with local devices and/or systems.

The embedded system of an IA is also typically configured with an actuator (wireless, contact, mechanical, optical etc.)—not shown, to actuate, activate for example, an onboard power source and in turn activate, power embedded circuitry.

IA control circuitry is configured fit-for-purpose to autonomously execute and enforce, and in general operate according to rules, rights, algorithms, functions, computational methods, variables, constants, data, predetermined conditions etc. The rules, rights, predetermined conditions etc. may be loaded/stored as needed and/or selected from among those previously stored in the IA (e.g., as software or firmware). Along with embedded firmware and hardwired primitives and functions, computational means etc., they determine the operation of the IA.

Importantly, an IA may be configured to generate different kinds of related or unrelated determinations, measures and evaluations; make decisions and perform actions, based on different sensed/perceived conditions, computational means/methods and predetermined conditions/criteria. For example, one measure may be used to alert, inform patients and caregivers as to a medication's fitness-for-use, while another measure (e.g., the contribution of custodian to an item's CET or change in fitness-for-use, while it was in their custody), may be used to evaluate, and take actions to motivate custodial stakeholders to improve their respective performances, e.g. generate trustworthy ‘proofs’ of their compliance with storage and handling protocols, that enable pay-for-performance.

An IA may also be advantageously configured with ad hoc power enabled (AHPE) circuitry: e.g., AHPE wireless communication circuitry, AHPE sensing circuitry (e.g., that comprises temperature actuated electrical (TAE) switches), or AHPE fail-safe systems—each of which are described in patents, patent applications or provisional patent applications at the end of this disclosure, and incorporated herein by reference.

While the focus herein is on medications, it is to be understood that IA's, and their related systems and methods described herein, may be configured for use with a wide variety of items that are sensitive to temperature and/or other conditions (or combinations of conditions) such as the absence, presence or the magnitude of, or change or rate of change in, humidity, moisture, shock, vibration, motion, centrifugal force, tilt, pressure (atmospheric, mechanical; weight), oscillation, acceleration or deceleration, electromagnetic radiation (visible light, infrared, ultraviolet, radio waves, microwaves, x-rays, gamma rays, acoustic radiation etc. Items may also be sensitive to the presence or absence of inorganic or inorganic materials, substances, compounds, chemicals, liquids, gels, gasses etc. either ambient, or within the item (e.g., byproducts of other conditions)—which may be sensed by their properties or characteristics, e.g., optical (e.g., color, patterns, reflectivity, transparency), physical (e.g., hardness, density, porosity, homogeneous, heterogenous, separation, texture), electrical (e.g., resistance, conductivity), or odor.

While progressive sensing and subsequent determination of cumulative condition and related measures (e.g., an item's fitness-for-use), is advantageously performed over the useful life, lifetime or lifecycle of the item, performances over shorter periods may however still be useful. For example, when a medication (e.g., pills) is packaged (placed in a pill bottle) and an IA affixed to the package at a pharmacy (vs. at the point of manufacture) which then progressively senses and determines the medication's CET or fitness-for-use until it's expiration date or it is determined to be no longer fit-for-use (e.g., at patient's home).

IA's operate autonomously over time according to predetermined conditions analogous to the terms and conditions of legal contracts. They determine (govern) how individual IA's perform and act on behalf of stakeholders in the item to which they are physically associated and its use, outcomes, purpose. And they are the key to trust in the performance and actions, and the results and outcomes, of IA's—and thus dependent actions and transactions involving custodial and noncustodial, local and remote, human and machine, stakeholders (parties). Accordingly, fit-for-purpose IA's can perform and act as agents on behalf of parties to contracts (legally binding and otherwise), and even to enter into contracts with other parties (e.g. via electronic contracts, smart contracts, other IA's).

### Exemplary Intelligent Agent Processes

Described below are exemplary IA processes. They may be activated, initiated, or triggered (“activated”):
in different combinations or sequences
in response to different conditions, events, actions, triggers
one or many times (or not at all) over the lifetime of an item

Each type of IA process may comprise multiple variations, e.g., a Sense Condition process may for example, use different sensing circuitry for different ambient conditions (e.g., temperature and humidity), and they may be executed in response to different triggers. The trigger and frequency, precision, accuracy, duration etc. of an IA process may be pre-set and/or dynamic; that is it may be modified or adapted (according to the current instance of predetermined conditions), in response to one or more events, changes in conditions, other processes and their results and outcomes, etc.

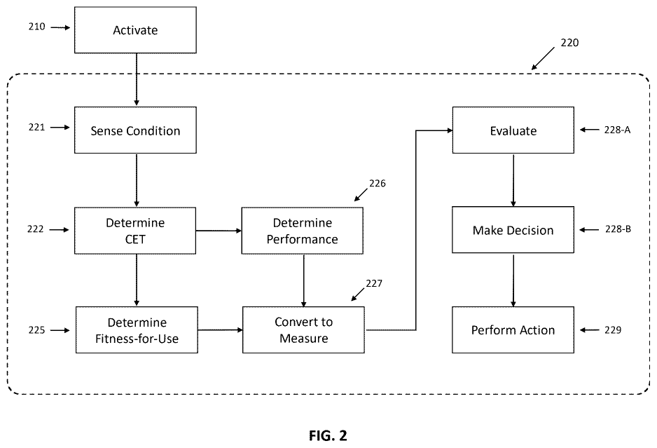

Note that IA processes may be implicitly or explicitly combined. For example, instead of a first process for evaluating a computed ‘measure’ of an item's fitness-for-use, and a second process using the results of the evaluation to make a decision, a single process may make a decision using the measure of fitness-for-use, without explicitly evaluating the computed measure. Thus by way of illustration in FIGS. 2-6, Evaluate process is numbered 228-A and Make Decision process is numbered 228-B. Note as well, that FIGS. 2-6 illustrate exemplary IA processes and relationships between them, which are to be understood as not being limited to those illustrated.

In general, an IA process may be configured to be activated, initiated or triggered in response to events, predictive algorithms/computations, other IA processes including, but not limited to, evaluations, decisions made, and actions taken. Importantly, IA processes, alone or in combination, may be iterative and the progressive results used to modify, adapt, optimize operations/IA processes, predetermined conditions etc. according to, and as allowed by, the current instance of predetermined conditions (and smart contracts as described for IA hardware agents in the section IA Hardware Agents).

IA's may be configured (with appropriate sensing and control circuitry) to perform IA processes that go beyond those related to the efficacy of an item, and configured to perform processes related to item's utility that are independent of it's efficacy e.g.,
byproducts of reactions to ambient conditions over time, such as their toxicity or gaseous emissions (safety), or
changes in their perceptual state (color, texture, separation, acidity, taste etc.)
   Such changes in an item's utility can:
reduce the perceived value of an item
generate false positives or false negatives, uncertainty as to its fitness-for-use, fitness-for-purpose, which lead to waste and in the case of medications for example, may lead to anxiety, disruptions in therapy and misdiagnosis.

Now described and illustrated in FIG. 2 are exemplary IA processes 220 for an IA configured to operate according to predetermined conditions once activated 210. Medications are the exemplar item, and ambient temperature the exemplar condition.

### Sense Condition Process 221

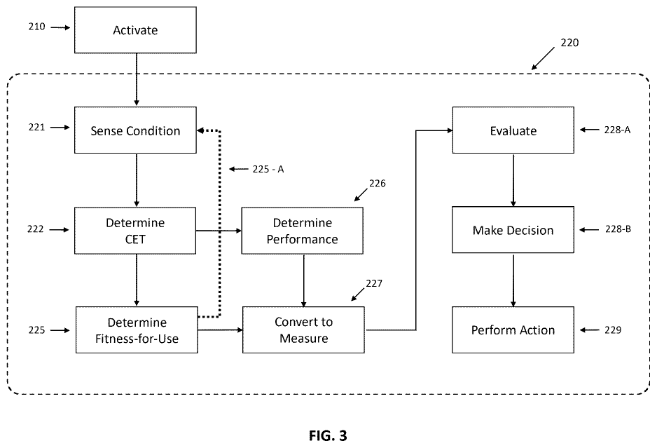

When activated, Sense Condition process 221 senses and generates values of the current instance of one or more conditions (e.g., ambient and/or those of a container, package of an item, or of the item itself). Sense Condition process 221 may be activated, initiated or triggered, adapted or modified, according to predetermined conditions, by one or more events or processes such as:
Initial activation of the IA embedded circuitry-Activation 210
One or more conditions, or changes in conditions (sensed and/or ad hoc power enabling as described in U.S. patent application Ser. Nos. 18/431,999 and 18/742,852, both entitled Ad Hoc Power Enabled Sensing Devices)
   symmetric and asymmetric
   in-bounds, out of bounds (excursions)
   magnitude, amplitude, duration, rates of change etc.
Time or changes in time (actual, relative)
   fixed (for one or more periods, intervals)
   time of day
   elapsed
      from initial actuation or subsequent activation of embedded circuitry
      during a fixed interval/period
      time remaining until a predetermined expiration date/end-of-life
Changes in location (e.g., with location sensing or receipt of a signal), custody or transport
Age, expiration status, level of stored energy-projected battery life, (per U.S. patent application Ser. No. 18/610,508 entitled Ad Hoc Power Enabled Fail-Safe Systems)
External signals/communications
   From a user, monitor/sensor, computer or network system etc.
In response to, using the results of, other processes as described above in general, and illustrated in FIG. 3 as described for Determine Fitness-for-Use process 225 below.

### Determine CET Process 222

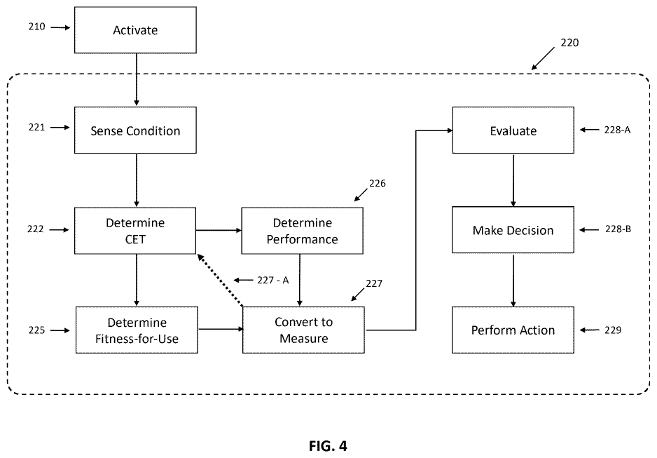

Determine CET process 222 determines values of CET using the results of Sense Condition process 221. A preferred embodiment of Determine CET process 222 computes mean kinetic temperature (“MKT”) and is described in detail in the section Exemplary Intelligent Agent-Mean Kinetic Temperature (“MKT”). Determine CET process 222 may be activated as described above in general, and illustrated in FIG. 4 as described for Convert to Measure process 227 below.

### Determine Fitness-for-Use Process 225

Determine Fitness-for-Use process 222 determines values of an item's fitness-for-use (it's utility) using values of CET determined by Determine CET process 222. Determine Fitness-for-Use process 225 may be activated automatically in response to determination of a new current instance of CET, and in response to, using the results of, other IA processes such as those described above. FIG. 3 illustrates (via dotted line 225-A) an exemplary Determine Fitness-for-Use process 225, whereby the result of a determination, e.g., absolute value, incremental change, or rate of change etc., automatically modifies (increases or decreases) the frequency of Sense Condition process 221, and in turn Determination of CET process 222 and Fitness-for-Use process 225 (not shown).

### Determine Performance Process 226

Similar to Determine Fitness-for-Use process 225, Determine Performance process 226 determines values using values of CET determined by Determine CET process 222. The predetermined conditions however are different, and the values determined by Determine Performance process 226 and their derivatives, are indicative of, relate or are specific to, the intended and actual performances of custodial stakeholders. And for example, they can be used to evaluate the ‘performances’ (actions, outcomes) of custodial and noncustodial stakeholders, e.g., those that affect the condition, utility, use or outcome of a medication such as compliance with handling and storage protocols, using or reporting fitness-for-use or expiration information (e.g., that generated by the IA). And further, wherein local and remote users, stakeholders, systems (including those that set the predetermined conditions upon which the IA operates) can use the determined performances and related data and information (advantageously in the form of ‘proofs’), to perform evaluations, actions or execute transactions accordingly (e.g., pay-for-performance, distribution of costs and benefits among the stakeholders).

### Convert to Measure Process 227

Convert to Measure process 227 converts determined values of CET, Fitness-for-Use or Performance (and fit-for-purpose, their determinants or derivatives, and/or results of other IA processes and related data and information) into ‘measures’ meaningful or purposeful to, easily communicated to, and understood and trusted by, local and remote, custodial and noncustodial, “stakeholders” in an item (medication) and its uses or outcomes (e.g., patients and caregivers, clinicians, healthcare providers, pharmaceutical companies, payers, etc.); evaluated by Evaluate process 228-A (or used by other IA processes (below). Measures (and their determinants and derivatives, and the determinations of other IA processes) may be determined using simple indexes, scales, lookup tables etc., or more complex computing means and the methods.

FIG. 4 illustrates (via dotted line 227-A) an exemplary Convert to Measure process 227, whereby a determination of fitness-for-use and a determination of performance (generated by Determine Fitness-for-Use process 225 and Determine Performance process 226 respectively) are converted and according to the resultant measure, CET process 222 is automatically activated (initiated), and in turn so are Determine Fitness-for-Use process 225 and Determine Performance process 226 respectively. The cycle then continues until the measure generated by Convert to Measure process 227 meets predetermined conditions for use by other IA process—e.g., Evaluation process 228-A, Make Decision process 228-B and Perform Action process 229 according to predetermined goals (conditions).

### Evaluate Process 228-A

Evaluate process 228-A evaluates determined measures and current (or past) instances of sensed conditions, their determinants and derivatives, the results of other IA process-including previous evaluations, decisions and actions, alone or in combination (not shown). And further, fit-for-purpose using related data and information such as (for a medication):
Patient sensitivity to, dependency on, the medication's fitness-for-use (relative risk, consequences, urgency)
Pending expiration, expiry status, date (preset, dynamic/projected), replacement options
Storage and handling protocols
Other prescribed medications, contraindications (accessed ad hoc from other IA agents, local and remote systems etc.)
   As described above and below, the results of Evaluate process 228-A can be used by the IA to adapt or modify, activate, initiate or trigger other IA processes as described above and later for other IA processes and illustrated in FIGS. 3 and 4.

### Make Decision Process 228-B

Using an evaluation from Evaluate process 228-A alone or in combination with determinants or derivatives of the evaluation, and/or with the results of other IA processes (and their determinants or derivatives), Make Decision progress 228-B makes a decision for Perform Action process 229 to take an action (or not). Make Decision progress 228-B may also make decisions without an evaluation, using the results of other IA Processes such as sensed conditions, determinations of CET, fitness-for-use or performances.

### Perform Action Process 229

In response to an explicit or implicit decision made by Make Decision process 228-B, (or another other IA process), Perform Action process 229 performs one or more actions and/or executes transactions (“actions”) using stored data, information, rules, rights and permissions, including the results, determinants and derivatives of IA processes 220 (and others), according to predetermined conditions. Exemplary actions (and transactions) include:
Storing, recording, packaging, generating proofs of:
   data/information related to, generated by, or a derivative or determinant of, IA processes
   interactions with (or transactions involving) users, local devices (including other IA's), networks, remote computer systems, stakeholders etc.
Messaging, using the messaging circuitry (of one or more types)
Communicating using the communication circuitry
Activating, initiating, adapting or modifying
   sensing and/or other IA processes, or
   predetermined conditions, rules, rights of the stakeholders etc. (as allowed by the current instances of them)

### IA Hardware Agents

An IA may be configured as a “hardware agent” as described in U.S. patents, pending and provisional applications. . . . As a hardware agent, an IA operates autonomously according to a progressive smart contract (advantageously a smart subcontract descended from a master contract), stored in its memory that comprises:
an electronic contract (comprising predetermined conditions) that determines the autonomous operations/performance of the IA agent, and
an electronic registry of stakeholders (comprising their rights, permissions etc.),
wherein the smart subcontract automatically executes, controls or documents events or actions according to the current instance of the electronic contract (and predetermined conditions), and
wherein the current instance of the electronic contract and the current instance of the electronic registry, are in whole or in part, immutable except as allowed by the current instance of the electronic contract and the current instance of the electronic registry.

In the broadest sense of the term, an IA on its own, or as a hardware agent, may comprise artificial intelligence (“AI”), and as described herein, perform operations and processes such as:
perceiving and input processing
evaluating, reasoning (‘determining’)
decision-making
executing actions, responding
learning, adapting
   An IA may therefore be an AI agent:
simple, primitive or complex
simple reflex, model-based reflex, goal-based, utility-based, hierarchal etc.

IA's may be configured to operate within local area mesh networks. Systems of IA's may further comprise local and/or remote computers (computing devices) or systems (centralized, decentralized, distributed) and many stakeholders. Advantageously such computers and systems are configured to perform actions or execute transactions using, or in response to, data and information (and proofs) from IA's:
Generated by one or more IA processes 220 or others
Used, transformed, derived from data/information or actions of an IA(s) e.g., from
   a local user (mobile phone, tablet, notebook computer), local ‘reader’/wireless station (storage, transport etc.;
   a receiving depot or POS terminal, —when for example an expired/medication—one no longer fit-for-use, is returned for replacement and/or disposal.

Such computers/systems may use learning systems, artificial intelligence and data/information/proofs from IA's, alone or together with related data/information (e.g., related patient healthcare outcomes). And advantageously do so with knowledge of the predetermined conditions with which the data/information was generated, to, for example, optimize:
Predetermined conditions and processes (computational means and methods, data, rules, rights, embedded firmware, hardwired primitives and functions etc.) by which individual, groups or classes of IA's operate
Practices, procedures, protocols, actions of, or related to stakeholder performances (and their evaluation, and compensation accordingly).
The allocation of costs and benefits among the stakeholders (and the computational models used to determine them)
Development of medications and therapies, or more generally items and their application, uses—utility and corresponding prices
Distribution channels, transportation methods and routing
User, stakeholder performances and outcomes (e.g., those of patients and their healthcare)

### Exemplary Intelligent Agent—Mean Kinetic Temperature (“MKT”)

Described now is an exemplary IA configured with temperature sensing circuitry and control circuitry to progressively determine a medication's CET throughout it's lifecycle (useful life, lifetime), wherein the determined (computed) CET is the medication's Mean Kinetic Temperature (MKT) which correlates to it's fitness-for-use. MKT is a computed temperature where the level of degradation over a certain period is the same as the total of separate degradations that could result from a series of different temperatures throughout the same total time period, respectively (see reference below). Although not illustrated, MKT can therefore be used in the determination and evaluation of performances.
United States Pharmacopeia and National Formulary (USP 43-NF38). Rockville, MD: United States Pharmacopeia Convention; 2020: General Chapter—Good Storage and Distribution Practices.

MKT has been used in the development of guidelines and protocols for handling and storage of temperature sensitive medications and monitoring compliance with them. An example of the former is “controlled room temperature” which the United States Pharmacopeia and National Formulary describes as a temperature that includes the typical environment of 20° C. to 25° C. (68° F. to 77° F.), where excursions are allowed between 15° C. and 30° C. (59° F. and 86° F.) provided that the MKT remains at or below 25° C. (see reference below).
United States Pharmacopeia and National Formulary (USP 43-NF38). Rockville, MD: United States Pharmacopeia Convention; 2020: General Chapter-Packaging and Storage Requirements.
   Note that MKT can be used with a wide range of items, such as those disclosed in the section below entitled Beyond Medications. Also, fit-for-purpose, other algorithms and computational models may be used for temperature, and of course other conditions as described later.

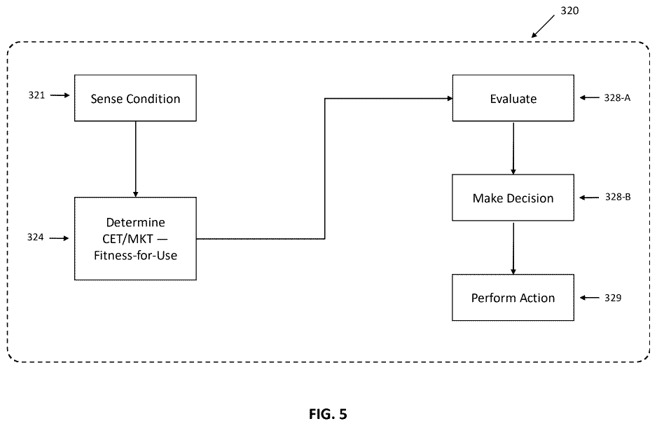

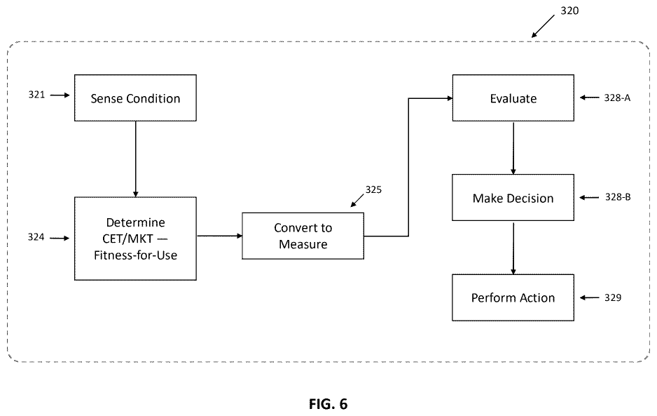

Through the selection of variables (predetermined conditions) used in the computation of a medication's MKT, the results of the computation (a determination of MKT) express the medication's fitness-for-use. As illustrated in FIG. 5 a computed MKT is both a determination of CET and a determination of Fitness-for-Use and can computed by the same CET/MKT-Fitness-for-Use process 324, and then directly evaluated by Evaluate process 328-A. The results of the evaluation and subsequent decision made by Make Decision process 328-B, may then be enacted by Perform Action process 329. Advantageously as illustrated in FIG. 6, the results of CET/MKT-Fitness-for-Use process 324 can be converted into a measure via Convert to Measure process, then evaluated with Evaluate process 328-A.

### Mean Kinetic Temperature (MKT)

MKT is based on the Arrhenius model for reaction kinetics. The Arrhenius equation (Equation 1) is described below:

$$k = {Ae}^{{(}^{- Δ H / RT} )} \qquad Equation 1.$$

### Where:

k is the reaction rate constant.
A is the frequency or pre-exponential factor.
ΔH is the activation energy (in kJ mol⁻¹)
R is the gas constant (in J mol⁻¹ K⁻¹)
e(−ΔH/RT) represents the fraction of collisions that have enough energy to overcome the activation barrier at temperature T.

The exponential term in the Arrhenius equation implies that the reaction rate increases exponentially when the activation energy increases, thus small changes in temperature can have a large impact on the reaction rate of a decaying/decomposing substance.

The MKT equation (Equation 2 below) utilizes Arrhenius model to compute the effective temperature where the level of degradation over a certain period is the same as the total of n separate degradations computed individually.

$$MKT = \frac{( Δ H / R )}{( - ln ( \frac{{t}_{1} e {(}^{- Δ H / {RT}_{1} )} + {t}_{2} {e}^{( Δ H / {RT}_{2} )} + {t}_{3} {e}^{( - Δ H / {RT}_{3} )} + … ... . + {t}_{n} {e}^{( - Δ H / RTn )}}{{t}_{1} + {t}_{2} + {t}_{3} + {t}_{4} + {t}_{5} + {t}_{6} + … ... ... .. + {t}_{n}} ) )} \qquad Equation 2$$

### Where:

MKT is the mean kinetic temperature in kelvins.
ΔH is the activation energy (in kJ mol⁻¹)
R is the gas constant (in J mol⁻¹ K⁻¹)
T₁ to Tₙ are the temperatures at each of the sample points in kelvins.
t₁ to tₙ are time intervals at each of the sample points.

MKT can be used with the classic rate equation (Equation 3 below) to determine the decay of the therapeutic agent (and by extension, the medication) over time.

$$\frac{- d [ B ]}{dt} = {k [ B ]}^{β} \qquad Equation 3$$

### Where:

B is the reactant.
β is the reaction order.
K is the reaction rate constant as computed using manufacturer's stability testing or labelled data that includes the medication's expiration date, maximum controlled room temperature during manufacturer's stability testing, and if available, expected viability or fitness-for-use at labelled time of expiration.

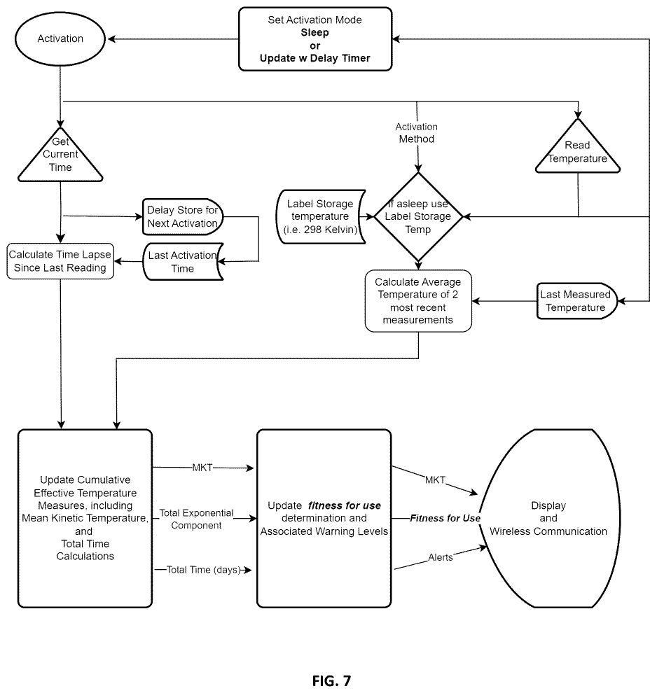

FIG. 7 provides a flow diagram of an exemplary implementation of an IA system for providing a temperature-based viability assessment of a therapeutic agent. Process steps for the exemplary implementation are now described:
Activation (as the term is used in this context): Each IA process cycle begins with Activation. Activation can be triggered by multiple processes including but not limited to:
   Timed event related to time of day.
   Fixed update interval.
   Adaptive update interval based upon current temperature, remaining battery life, remaining time until expiration date.
   Activated in response to an external event, or change in condition. Essentially the IA is set to sleep and/or be awakened by an external trigger/event. During sleep mode, the IA has minimal energy requirements prolonging battery life. Activation triggers could include:
      Changes in temperatures or other ambient conditions (as described elsewhere herein); e.g., across predetermined boundaries/thresholds as exemplified in U.S. patent application Ser. No. 18/431,999 entitled Ad hoc Power Enabled Sensing.
      Breakdown of product packaging (i.e. microplastics)
      Air (or ingress of any chemical) either stored within the packaging or diffused through the packaging, or when the packaging is intentionally or accidentally opened.
      Time-related changes in any of the external conditions.
      Communications from/with users/parties (e.g., using mobile phones) local devices, systems and networks.
      Combination of events/conditions such as those listed above. One example is to have a maximum time interval set (i.e. 1 day), but the system wakes up if temperature exceeds label maximum controlled temperature. During this period, the interval is adaptively decreased until the temperature falls again to below the maximum labelled control temperature.
The activation interval (time between activations) must balance accuracy and precision of the measurements with remaining battery life and time until expiration date. Knowing from Arrhenius model for reaction kinetics that viability decay rate increases exponentially with temperature, shorter activation intervals and/or tighter tolerances around change in temperature could be implemented to better balance accuracy. In addition, some substances may have boundary limits (i.e. 0° C.) wherein the substance degrades very quickly, hence activation interval during the episodes should also decrease accordingly as temperature approaches these boundary conditions. Activation also sets flag, Activation Method, to identify trigger used to initiate the current cycle.
Get Current Time: Read the current time Timeₙ from the IA circuitry (or other source). Time is an important component of determining MKT, Substance viability, and remaining time until expiration.
Calculate Time Lapse Since Last Reading: Calculate Time Interval tₙ from MKT computation in Equation 2, using current time and time stored from last activation tₙ=Timeₙ−Timeₙ₋₁.
Delay Store for Next Activation: Stores current time in buffer for next activation cycle Timeₙ₋₁ to compute time interval between activations.
Last Activation Time: Accesses stored time Timeₙ₋₁ from memory for time interval computation.
Read Temperature: Acquires temperature from sensor Temperatureₙ from MKT computation in Equation 2. Other sensed conditions/states may be acquired at this time for processing. Temperature reading is converted into units of Kelvin for computation of MKT.
Label Storage Temperature: Stored input value specific to Manufacturer and pharmaceutical agent that describes the controlled room temperature restrictions in Kelvin over which shelf life was computed.
Switch If Asleep Use Label Storage Temperature: Uses the Activation Method flag to determine which Temperature value is used in current update. If the system had been asleep then Temperatureₙ is set to Label Storage Temperature, else Temperatureₙ is set to current Temperature measurement from the sensor.
Last Measured Temperature: Stores current temperature in buffer for next activation cycle, and subsequently accesses stored temperature Temperatureₙ₋₁ from memory for average temperature computation over the recent time interval tₙ.
Calculate Average Temperature of 2 Most recent Measurements: Calculates the average of Temperatureₙ and Temperatureₙ₋₁ to determine the mean temperature Tₙ corresponding to tₙ.
Update Mean Kinetic Temperature and Total Time Calculations: Utilizes the recent temsperature and time interval data to update computations for the remining shelf life. For the basic temperature based MKT system, FIG. 8 provides a flow diagram of the computational process. This process can be extended to additional inputs and computation methods to determine the cumulative effect of external conditions (temperature, pressure, light, humidity, packaging integrity, etc. along with their 1^{st} and 2^{nd} derivatives) on the viability individually and collectively. One such method is to compute total time that control substance exceeds limit ranges established by manufacturer as a check for viability of the therapeutic agent.

Outputs (results) from the above processes can include cumulative time, MKT, Total exponential component of the MKT equation. These values can be stored and accessed by the user to review data and identify periods wherein storage conditions did not meet requirements in order to implement better supply change controls.
Update Fitness-For-Use Calculation and Associated Warning Levels: This process accepts computations from Update Mean Kinetic Temperature and Total Time Calculations process and computes the Fitness-for-Use of the therapeutic agents and provides information in useable framework. Calculations may include assumptions as to how the substance is maintained moving forward. Output from this process could be in many forms, depending on the information desired to be messaged/communicated to the user. The results/output of this process could include:
   A bichrome (or symbolic) visual alert that the medication is no longer viable.
   A plurality of visual indications (i.e. Red, Yellow, Green) that message different states or conditions, levels of urgency/calls-to-action. For example a Yellow indicator that alerts a medication is within X number of days of expiring under controlled room temperature conditions. Or, that the efficacy, fitness-for-use of the medication is decaying/degrading faster than called for by the labelled protocol (controlled room temperature).
   Mean Kinetic Temperature to date (the current/most recent determined MKT)
   Mean Kinetic Temperature estimate provided the medication is maintained per handling and storage protocols hereafter (e.g., controlled room temperature).
   Extreme fluctuations and duration including time and date information that may lead to better policies and procedures for manufacturers, distributers, providers and patients to better maintain medication viability.
Display and IA Monitor Communication: Information from the computations can be provided in several mechanisms including:
   Markers (Flags) on the medication labelling such a as a red X across the label if the medication is no longer viable.
   Analog strip that is filled in as viability limit is approached.
   Multicolor markers providing a more granular viability status update.
   Communication with the monitor providing detailed information as described in Update Cumulative Effective Temperature including MKT and Associated Warning Levels. More detailed information may be provided, segmented by time or event driven intervals. Data can be in the form of time synched arrays of MKT, exponential component of MKT, maximum and minimum temperatures and other parameters during time segments. Time synched information are intended to find gaps in in supply chain handling of material.

FIG. 8 provides a detailed description of the data flow diagram associated with Update Mean Kinetic Temperature and Total Time Calculations found in FIG. 7. In the exemplar the inputs to this process are temperature Tₙ and time interval tₙ.

Calculate New Exponential Component: The exponential component of MKT is updated using the current time interval tₙ and temperature measurements Tₙ.

$${MKT}_{n}^{exp} = {t}_{n} {e}^{( - Δ H / {RT}_{n} )}$$

### Where:

∇H is the activation energy in units of kJ mol⁻¹, defined as the minimum amount of extra energy required by a reacting molecule to convert into a product. Unless the stability data is available from the medication's manufacturer, a default value for the activation energy is set:

$$∇ H = 83.144 kj / mol$$

and
R is the molar gas constant. The constant is also a combination of the constants from Boyle's law, Charles's law, Avogadro's law, and Gay-Lussac's law. The gas constant is a physical constant expressed in terms of units of energy per temperature increment per mole. It is also known as the ideal gas constant or molar gas constant or universal gas constant. The gas constant value is equivalent to the Boltzmann constant but expressed as the pressure-volume product instead of energy per increment of temperature per particle.

$$R = 8.31446261815324 J / K \cdot mol$$

such that

$$- \frac{∇ H}{R} = - 9999.913404$$

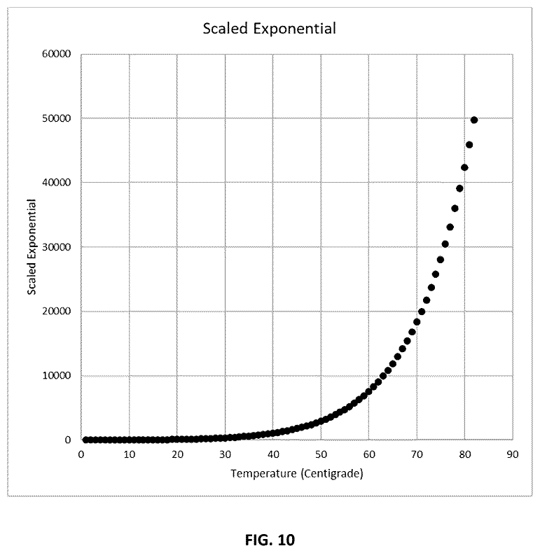

These equations are presented in floating point format. Under the limitations of IA's, the computations can be computationally simplified. One method is to implement a look-up table that directly transforms temperature Tₙ into e(−ΔH/RTₙ) as shown in. The transformed temperature can then be advantageously scaled to operate in fixed integer mode by subtracting e(−ΔH/R(273) from the result and multiplying by a scaling factor of 100000000000000000 as shown in FIG. 10. Scale factors can be optimized in concert with range-of-temperatures and precision requirements.

Update Total Time: Current time interval tₙ is added to existing total (or cumulative) time TotalTimeₙ to update the total time since manufacturing date of the substance. The computed result/value is used in computing MKT, and other parameters associated with medication viability.

$${TotalTime}_{n} = {TotalTime}_{n - 1} + {t}_{n} = {\sum }_{i = 1}^{i = n} {t}_{i}$$

Store Last Time Total: Stores TotalTimeₙ in memory for next iteration TotalTimeₙ₋₁ for updating the computation over the next cycle.

Update Total Exponential Component: The total exponential component of the MKT

$${TotalMKT}_{n}^{exp}$$

is updated each cycle iteratively from prior total exponential component

$${TotalMKT}_{n - 1}^{exp}$$

and the newly computed exponential component.

$${TotalMKT}_{n}^{exp} = {TotalMKT}_{n - 1}^{exp} + {t}_{n} * {MKT}_{n}^{exp}$$

Store Last Total Exponential Component:

$${TotalMKT}_{n}^{exp}$$

is stored in memory to be used in the next activation cycle.

Calculate Mean Kinetic Temperature (MKT): MKTₙ defined in Equation 2, can now be iteratively computed at each activation cycle from

$${TotalMKT}_{n}^{exp}$$

### and TotalTimeₙ.

$${MKT}_{n} = \frac{- ∇ H / R}{ln ( \frac{{TotalMKT}_{n}^{exp}}{{TotalTime}_{n}} )}$$

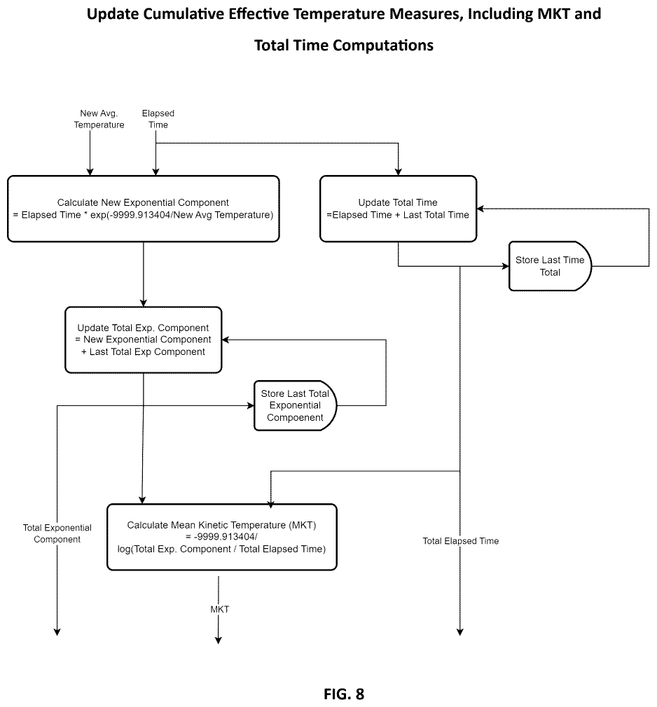

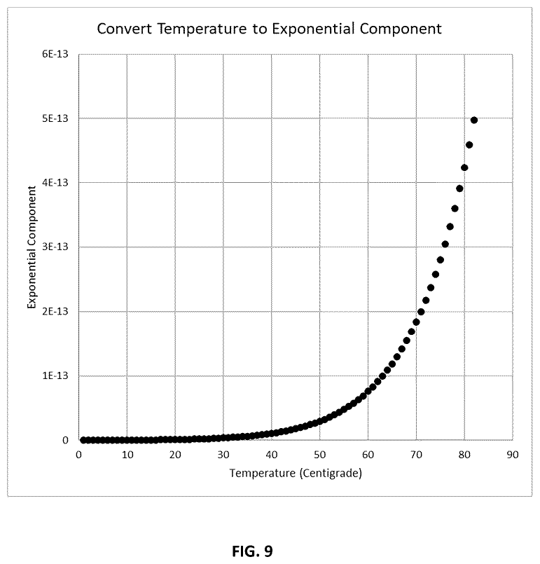

These equations are presented in floating point format. Under the limitations of IA, the computations can once again be computationally simplified. The method is to implement a look-up table that directly transforms temperature Tₙ into e(−ΔH/RTₙ) as shown in FIG. 9 and FIG. 10, and then can be reversed transforming the computed value back into temperature units of MKT.

Similar processes can be used to iteratively compute and update medication viability using additional data from other sensed conditions as previously described including their 1^{st} and 2^{nd} derivatives. In addition, the results of these computations/updates can be used in combination and as a function of time.

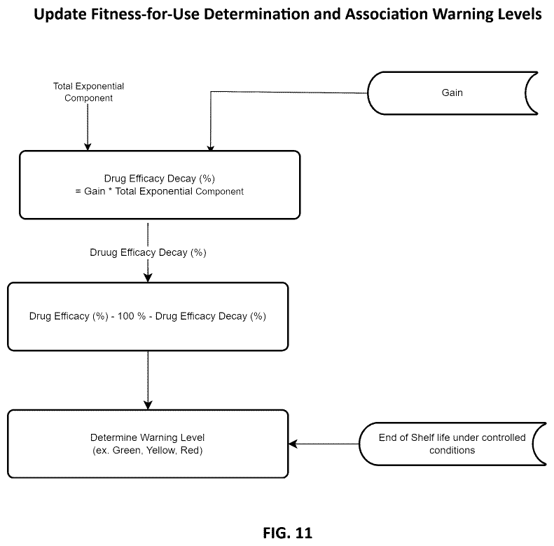

FIG. 11 provides a detailed description of the data flow diagram associated with Update Fitness-for-Use Determination and Associated Warnings found in FIG. 7. In the example, the input to this process is simply

$${TotalMKT}_{n}^{exp} .$$

Alternatively, Mean Kinetic Temperature MKTₙ and TotalTimeₙ can be used to perform computations.

Gain: Decay in Medication Efficacy can be computed is several ways depending on available information from the manufacturer. At a minimum, labelled shelf life and storage temperature ranges are provided. Under these conditions decay in medication efficacy is Efficacy Decayₙ defined from efficacy on day 0 (manufacturing date) as 0% to efficacy decay at labelled expiration date as 100% such that the equation bypasses the actual efficacy value (i.e. 90%) in the computations. The gain for the decay rate is computed using the expiration date and the maximum storage temperature.

$${Gain}_{x} = \frac{1}{Expiration Period * {e}^{{(}^{- Δ H / R * Max Storage Temperature in Kelvin} )}} * 100 \%$$

This value can be programmed into the IA specific to individual substances/medications. Alternatively, if the manufacturer provides the remaining efficacy, then the equation for GAIN can reflect that additional information.

$${Gain}_{e} = \frac{1}{Expiration Period * {e}^{{(}^{- Δ H / R * Max Storage Temperature in Kelvin} )}} * ( 100 \% - Efficacy at Expiration Date )$$

Medication Efficacy Decay: Utilizes the gain computed from the manufacturer's labeled shelf life (stability) testing and

$${TotalMKT}_{n}^{exp} ,$$

wherein

$$\% {EfficacyDecay}_{n} = {Gain}_{x} * {TotalMKT}_{n}^{exp} * 100 \% ,$$

and is iteratively updated each activation cycle.

Medication Efficacy: Calculates the remaining efficacy of the medication either overall or until it reaches the manufacturer's computed level at time of expiration, wherein % Efficacyₙ=100%−EfficacyDecayₙ, and is iteratively updated each activation cycle.

Determine Warning Level: The remaining efficacy is finally converted into a measure(s) for evaluation and subsequent action by the IA such as messaging with the user, or executing a transaction.

### Data/Information Storage

An IA can record/store data and information in a variety of formats and time durations, balanced against limitation on control circuitry, battery life and memory allocation. At one extreme, all temperature and time data are stored starting with the date of manufacture and continuing throughout the lifecycle of the medication. From these data, a local device, network or remote computer system or stakeholder can access and compute pertinent information. At the other end of the spectrum only the most recent data iteration is stored and subsequently accessible or messaged to users, or communicated to external entities. Intermediate methods of data storage may provide a balance between data utility and performance of the IA. Data can be segmented into prescribed intervals (days, weeks) or synched to events in the supply chain. Under the segmented storage approach, pre and post processed/analyzed data could be stored, e.g., time, time interval, mean kinetic temperature, temperature range, time outside controlled room temperature, exponential component of MKT computation, shelf life information. These values/measures can be computed and stored in time synched arrays for the defined intervals as a means of understanding temperature control throughout the supply chain.

### Extensions to MKT

The MKT exemplar describes a process(es) wherein temperature is the primary sensed condition. It also focuses on MKT as the primary method for computing/evaluating medication viability. Other processes and computational means exist in addition to those described elsewhere herein (and may be used alone or in combination).

The MKT exemplar also assumes that the decay rate in reference to temperature is zero order with time. This means that elevated temperature changes are independent of time of occurrence. Some medications can have a time-dependent degradation (1^{st} or 2^{nd} order with time) wherein temperature changes near the end of the shelf life may have a more profound impact than those early in the life cycle. The computation of MKT, including the degradation rate constant, can be adjusted to include 1^{st} and/or 2^{nd} order time dependent degradation.

Data Subset analysis: Data Subset analysis: The CET/MKT process(es) previously described include updating computations starting on the manufacturing date that produce mean kinetic temperature, temperature range, time outside controlled room temperature, shelf life information etc. Raw Time and Temperature data for example can be advantageously stored for the entire lifecycle (useful life) of the medication, or combined into predetermined intervals (e.g., Daily, Weekly) or synched to events or custodies along the distribution chain, and/or converted into more useful measures (e.g., MKT or temperature, cumulative duration wherein temperature exceeds given limits). These additional measures can subsequently used to identify adherence (or lack/thereof) to handling, storage and other protocols by stakeholders in the supply chain.

Conditions other than temperature can be used with the MKT model and the results used as feedback to determine the fitness-for-use, the shelf life or other state of a medication (or derivative thereof). Fitness-for-Use can be computed using additional data in combination with MKT, for example, using cross-terms and/or layered models described below. In addition to those described elsewhere herein, are for example, the:
Impact of temperatures beyond the conditions where MKT is primary driver for medication degradation. The condition, state or properties of medications can change symmetrically or asymmetrically in response to changes in temperature, humidity etc. For example, biologics may lose viability if the temperature falls below “low” boundaries/thresholds for short periods of time (due to phase changes), while it may take considerably longer if temperatures rise above “high” boundaries/thresholds. Hence strict limits may be placed on the duration that a sensed value exceeds different limits.
Rate of change or acceleration in the rate of change of temperature (1^{st} and/or 2^{nd} derivative of temperature) or other sensed condition.
Conditions wherein packaging material may deteriorate or change properties (ingress or diffusion properties).
Medications at risk of degradation due to phases change due to temperature changes (i.e. melt or freeze).
Medications at risk of degradation due to light (UV) exposure.
Medications at risk of degradation due to exposure to moisture.
Vacuum sealed packages, wherein the shelf life after opening is much shorter and under tighter restrictions compared to vacuum sealed packages.

### Exemplary Intelligent Agents—Beyond MKT

Controlled Circuit Logic: The MKT example describes processes wherein temperature is the primary sensed condition. It also focuses on MKT as a metric. Other exemplary IA's are configured wherein the control circuitry (including software, firmware,) can transform data of a single sensed condition in multiple ways, creating (computing) determinations of fitness-for-use(s) for each and then combining them into a single determination of Total Fitness-for-Use (or combine the data to compute the Total Fitness-for-Use using single more complex set of equations). Furthermore, this approach can be implemented using data of multiple sensed conditions, and/or inputs from the user or external device. The control circuitry can be configurated in various ways to combine information depending on the inputs and algorithms such as:
Logic Equations: A series of logic gate (“AND”, “OR”, “NAND”, and “NOR” are used to combine information.
Summing or Averaging: A straightforward process with minimal computational requirements for combining multiple criteria for fitness-for-use into single result.

$$TotalFitnessForUse = \frac{{\sum }_{i = 1}^{n} {FitnessForUse}_{i}}{n}$$

Weighted Summing: A straightforward process with minimal computational requirements for combining multiple criteria (predetermined conditions) for fitness-for-uses into single result, wherein one or more individual components are weighted more heavily than other components.

$$TotalFitnessForUse = \frac{{\sum }_{i = 1}^{n} {Weight}_{i} * {FitnessForUse}_{i}}{{\sum }_{i = 1}^{n} {Weight}_{i}}$$

Multiplication: A straightforward process with minimal computational requirements for combining multiple criteria for fitness-for-uses into single result. Multiplication processes operate akin to analog “AND” functions where the low/lower scores/results tend to dominate.

$$TotalFitnessForUse = {\prod }_{i = 1}^{n} {FitnessForUse}_{i}$$

Weighted Multiplication: A process for combining multiple criteria for fitness-for-use's into single result is implemented. To provide flexibility regarding limits and weights. This can be accomplished by incorporating a sigmoidal function that possesses minimum and maximum output values.

$$TotalFitnessForUse = {\prod }_{i = 1}^{n} {Min}_{i} + {Gain}_{i} / ( 1 + {e}^{- {Sensitivity}_{i} * ( {FitnessForUse}_{i} - {ExpOffset}_{i} )} )$$

Where:
   Minᵢ describes the minimum transformed value from FitnessForUseᵢ.
   Minᵢ+Maxᵢ describes the maximum transformed value from FitnessForUseᵢ.
   ExpOffsetᵢ describes the FitnessForUseᵢ value at which the output=Minᵢ+(Minᵢ+Maxᵢ)/2
and Sensitivity; describes the slope of the transformed value at the midpoint as a function of FitnessForUseᵢ.
Rule based or knowledge-based system: A system in which domain-specific knowledge is represented in the form of rules and general-purpose reasoning is used to solve problems in the domain. A typical rule-based system (process) has four basic components:
   A list of rules or rule base, which is a specific type of knowledge base.
   An inference engine or semantic reasoner, which infers information or takes action based on the interaction of input and the rule base. The interpreter executes a production system program by performing the following match-resolve-act cycle:
   Temporary working memory, which is a database of facts.
   A user interface or other connection to the outside world through which input, and output signals are received and sent.
A set of rules can be implemented to convert fitness-for-use data from individual sensed data. Such rules could be adjusted according to the medication and available sensed information.
Fuzzy logic: Wherein many-valued logic in which the truth value of variables may be any real number between 0 and 1, is implemented to handle the concept of partial truth, where the truth value may range between completely true and completely false. The most well-known system is the Mamdani rule-based one. It uses the following rules:
   Fuzzify all input values into fuzzy membership functions.
   Execute all applicable rules in the rule base to compute the fuzzy output functions.
   De-fuzzify the fuzzy output functions to get “crisp” output values.
Artificial Intelligence: Wherein as sensed/CET data are collected; algorithms are optimized using artificial intelligence (see the section above entitled Exemplary Intelligent Agent Processes).
Data Transformation: Prior and post combining different inputs to the IA/control circuitry (communications from users, oracles, local devices etc.), sensed and/or other data, and/or determinations of CET and/or Fitness-for-Use or utility and related computed values, can be transformed to facilitate said combinations. Combinations of inputs include but are not limited to:
   Sigmoidal curves
   Logarithmic
   Exponential
   Limiting terms
   Discontinuities
   Saturation curves
Cross Terms: Wherein inputs to the IA/control circuitry can be combined, including post transformation into the equations. These combination data can include other sensed data and/or data inputs from users et al. regarding for example, substance, storage requirements, transportation requirements, geography, ambient conditions (temperature, humidity, altitude, etc.).
Layered Models: Wherein the individual control circuitry can be further combined through model layers that provide higher level interaction and combination. This higher level may be useful in transforming data into actionable information for users throughout the supply chain including transportation, storage, distributor, provider, and end user. Layered models may also provide control circuitry configured to select and optimize one or more specific processes/means for computing/determining Fitness-for-Use from input data. This approach can also allow an IA (wirelessly enabled) to be utilized with various medications, substances etc. under various conditions by allowing, with appropriate end-to-end security, a remote computer system/network (or stakeholder) and secure to provide/update specific information to the IA specific to the use case.

### Intelligent Agents With Predictive Control Circuitry

The control circuitry of an IA can be configured to use MKT and Time information to predict the remaining shelf life of a medication in time units (i.e. days remaining), that is to:
Compute a count of the estimated number of days of shelf life remaining if a medication is maintained within manufacturer's guidelines. This provides the manufacturer, user and/or healthcare provider a continuous update regarding the time usage of the medication and influence the supply chain algorithms.
Compute a count the estimated number of days of shelf life remaining if the medication's exposure to its current condition is maintained at its current level (temperature, light, humidity, etc.). This provides the user et al. with feedback as to the need to remedy any situation where for example, the:
   Remaining shelf life is tied to warning levels displayed or provided to the user
   Remaining shelf life (or a corresponding measure) may be messaged or wirelessly communicated, e.g., to:
      Notify or alert or alarm a user
      Enable third parties (particularly together with shelf life information from a plurality of IA's) to adjust production or inventories across multiple locations.

By means of example, exemplary flow processes are described in the following figures. Three separate scenarios are provided. For all the scenarios it is assumed that the labelled shelf life is 2 years (730 days), and the maximum temperature in the controlled scenario is 30° C. The model further assumes that the decay in efficacy (fitness-for-use, utility) is zero order with time. That means the impact of temperature changes on efficacy decay is independent of the period within the scenario period. The first scenario is a control scenario wherein the temperature is set to 30° C. for the duration of the scenario. The second scenario involves setting the temperature 2° C. above the labelled maximum control temperature at 32° C. The third scenario involves a ten-day temperature excursion to 40° C. starting on day 61. The temperature returns to baseline on day 70. This excursion occurs with a constant baseline of 30° C.

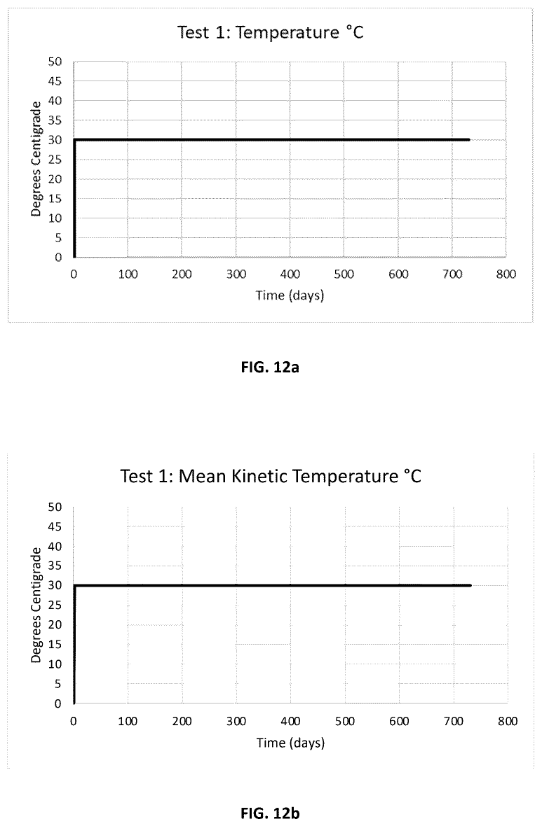

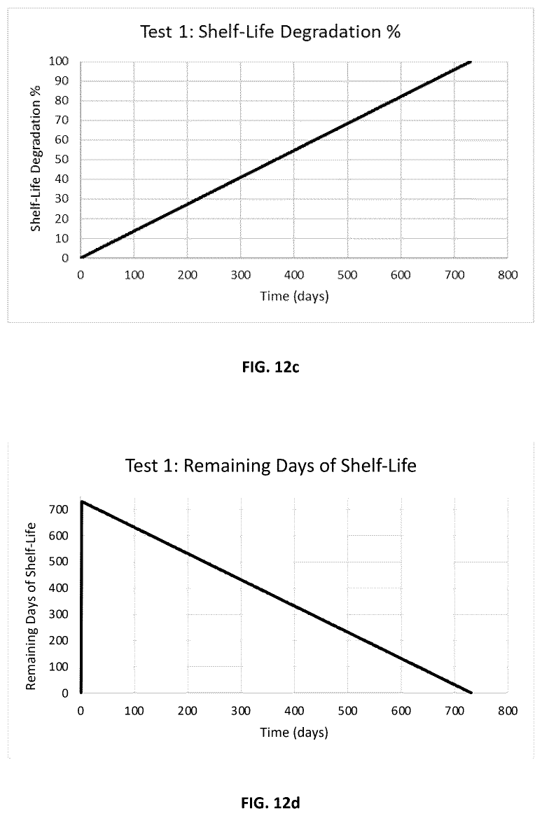

FIG. 12a shows the Temperature during the 730-day period for Scenario 1. The temperature is fixed at 30° C. throughout the scenario. Under these conditions the efficacy of the substance decays linearly throughout the 730 days reaching 100% at the labelled expiration date.

FIG. 12b shows the Mean Kinetic Temperature for Scenario 1. With a constant temperature of 30° C. across the entire scenario, the Mean Kinetic Temperature matches these values.

FIG. 12c shows the Shelf-Life Degradation ranging from 0% to 100% for Scenario 1. At day zero (Manufacturing Date) Shelf-Life Degradation is 0%. This reflects the fact that the substance has not yet begun to decay. With a constant temperature of 30° C. across the entire scenario, the substance degrades linearly with time so that at Shelf-Life date of 2 years, 730 days, Shelf-Life Degradation reaches 100%, meaning that the medication is no longer viable for administration.

FIG. 12d shows the Remaining Days of Shelf-Life for Scenario 1. On day zero (Manufacturing Date), the Remaining Days of Shelf-Life will equal the manufacturers labelled shelf life. This reflects that the substance has not yet begun to decay. With a constant temperature of 30° C. across the entire scenario, the substance degrades linearly with time so that at Shelf-Life Date of 2 years, 730 days, Remaining Days of Shelf-Life decreases to zero, meaning that the medication is no longer viable for administration. The slope of the line=(−1).

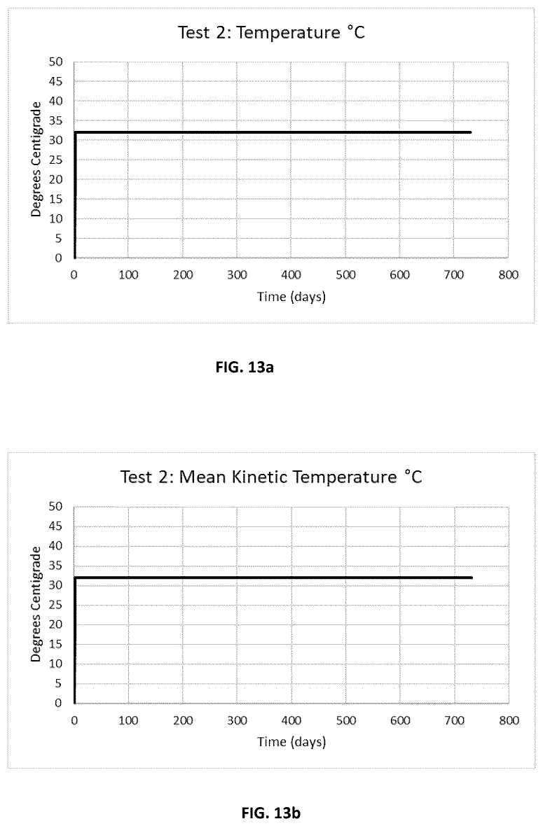

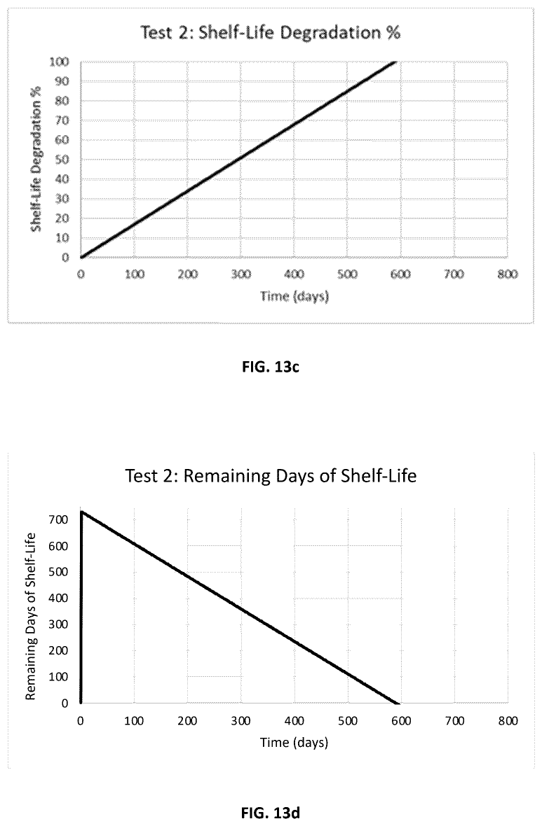

FIG. 13a shows the Temperature during the 730-day period for Scenario 2. The temperature is fixed at 32° C. throughout the scenario. Under these conditions the efficacy of the substance decays linearly throughout the 730 days reaching 100% prior to the labelled expiration date.

FIG. 13b shows the Mean Kinetic Temperature for Scenario 2. With a constant temperature of 32° C. across the entire scenario, the Mean Kinetic Temperature matches these values.

FIG. 13c shows the Shelf-Life Degradation ranging from 0% to 100% for Scenario 1. At day zero (Manufacturing Date) Shelf Life-Degradation is 0%. This reflects the fact that the substance has not yet begun to decay. With a constant temperature of 32° C. across the entire scenario, the substance degrades linearly with time so that at Shelf-Life date of 590 days Shelf-Life-Degradation reaches 100%, meaning that the medication is no longer viable for administration. 590 days is much shorter than the labelled 730 days, reflecting the negative impact of storage the substance outside labelled requirements.

FIG. 13d shows the Remaining Days of Shelf-Life for Scenario 2. On day zero (Manufacturing Date), the Remaining Days of Shelf-Life will equal the manufacturers labelled shelf life. This reflects the fact that the substance has not yet begun to decay. With a constant temperature of 32° C. across the entire scenario, the substance degrades linearly with time so that at 590 days, Remaining Days of Shelf-Life decrease to zero, meaning that the medication is no longer viable for administration. The slope of the line <(−1), showing that the shelf life is decaying faster than in Scenario 1, wherein the substance is maintained per label requirements.

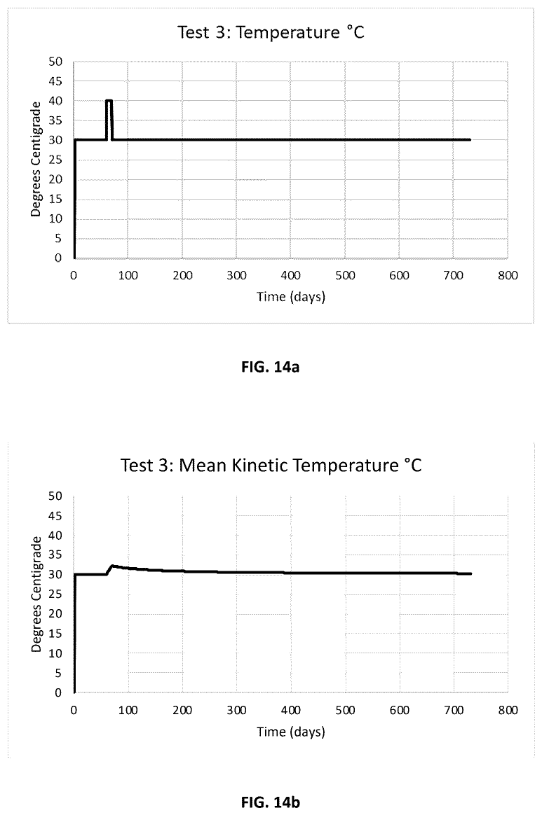

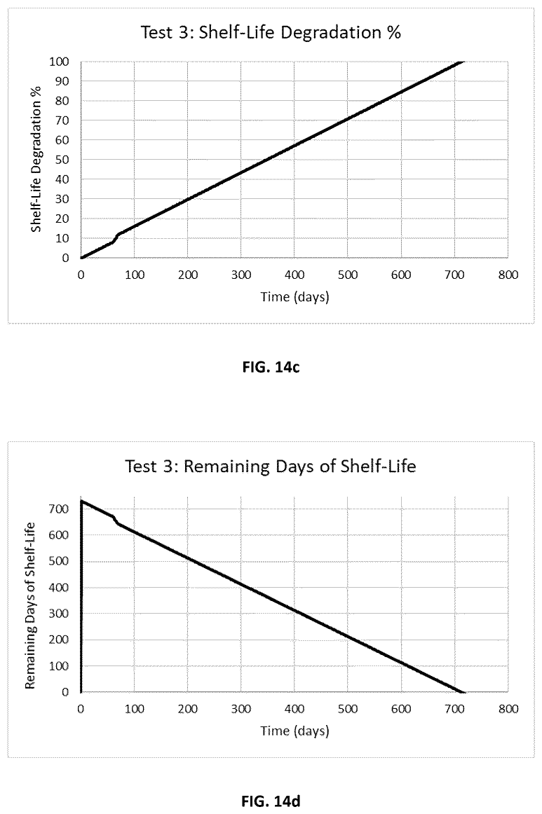

FIG. 14a shows the Temperature during the 730-day period for Scenario 3. The temperature is fixed at 30° C. for the 1^{st} 60 days. From day 60 through day 70, the temperature was raised to 40° C. From day 70 and throughout the remainder of the scenario, the temperature was once again set to 30° C. Under these conditions the efficacy of the substance decays more quickly during the period of 40° C. degree excursion, thus reaching 100% Shelf-Life degradation prior to the labelled expiration date.

FIG. 14b shows the Mean Kinetic Temperature for Scenario 3. With a constant temperature of 30° C. over the 1^{st} 60 days, the Mean Kinetic Temperature matches the 30° C. temperature for scenario 3. From day 61 to day 70, when the temperature is set at 40° C., The Mean Kinetic Temperature rises to 32.09° C. Note that 32.09° C. is higher than the average temperature of 31.4° C. When the temperature returns to 30° C. at 70 day mark, the mean kinetic energy temperature exponentially decays. At day 730, MKT=30.23° C., higher than the average temperature of 30.13° C.

FIG. 14c shows the Shelf-Life Degradation ranging from 0% to 100% for Scenario 3. At day zero (Manufacturing Date) Shelf-Life Decay is 0%. This reflects the fact that the substance has not yet begun to decay. With a constant temperature of 30° C. for the 1^{st} 60 days, the substance degrades linearly with time at a rate that matches the rate for scenario 1. At 60 days the decay rate increases reflecting the rise in temperature to 40° C. At 70 days, when the temperature returned to 30° C., the rate of shelf life degradation again matches the rate for scenario 1. Overall, the Shelf Life Degradation reached 100% at 710 days.

FIG. 14d shows the Remaining Days of Shelf-Life for Scenario 3. On day zero (Manufacturing Date), the Remaining Days of Shelf-Life will equal the manufacturer's labelled shelf life. This reflects the fact that the substance has not yet begun to decay. With a constant temperature of 30° C. for the 1^{st} 60 days, the substance degrades linearly with time at a slope of (−1), the same as scenario 1. At 60 days the Remaining Days of Shelf-Life decreases more dramatically, reflecting the rise in temperature to 40° C. At 70 days, when the temperature returned to 30° C., the substance degrades linearly with time at a slope of (−1), the same as scenario 1. Overall, the Remaining Days of Shelf-Life decrease to zero at 710, meaning that the medication is no longer viable for administration.

### Beyond Medications

The inventions described above are applicable to a wide range of items (properties, characteristics, and forms), packages and containers, such as:
Pharmaceuticals, antibiotics, vaccines, blood products, lab samples (for humans and animals)
Pills, injectables, aerosols, sprays, vaporizers, bandages and dressings
Topical agents (soaps, lotions, cremes etc.)
Supplements
Food, beverages
Cosmetics
   And in general, condition sensitive consumer and industrial materials, substances, chemicals, liquids, gasses; organic or inorganic; raw or processed; toxic, radioactive.

While particular preferred and alternative embodiments of the present intention have been disclosed, it will be appreciated that many various modifications and extensions of the above described technology may be implemented using the teaching of this invention. All such modifications and extensions are intended to be included within the true spirit and scope of the appended claims.

1. An intelligent agent physically associated with an item, comprising:
control circuitry, comprising in part predetermined conditions;
sensing circuitry for sensing an ambient condition or a condition of the item;
communication circuitry;
a power source; and
wherein the intelligent agent is configured to operate autonomously according to the control circuitry, and over time progressively execute the processes of:
sensing a condition;
transforming the sensed condition into a determination of the item's utility; and
according to the determined utility of the item, execute processes or perform actions.

2. The intelligent agent of claim 1, wherein the intelligent agent is affixed or bound to, embedded in or integrated with, the item or its packaging or container; or is configured for use with multi-item cartons, boxes, and other mobile or transportable containers

3. The intelligent agent of claim 1, further configured to operate over the useful life, life-cycle or lifetime of the item.

4. The intelligent agent of claim 1, wherein the control circuitry comprises:
a controller, logic, memory, a clock/timer, and
combinations of some or all of:
computing circuitry (primitives, functions, algorithms)
operating instructions, programs
predetermined conditions, rules, rights, and permissions.

5. The intelligent agent of claim 1, wherein the sensed condition is from among temperature, humidity, moisture, shock, vibration, motion, centrifugal force, tilt, pressure (atmospheric, mechanical; weight), oscillation, acceleration or deceleration, electromagnetic radiation (visible light, infrared, ultraviolet, radio waves, microwaves, x-rays, gamma rays, acoustic radiation).

6. The intelligent agent of claim 1, wherein the sensed condition is a property or characteristic of an organic or inorganic material, substance, compound, chemical, liquid, gel, or gas, of the item, a byproduct of the item, a contaminant, or the ambient environment.

7. The intelligent agent of claim 6, wherein the property or characteristic is optical (color, patterns, reflectivity, transparency), physical (hardness, density, porosity, homogeneous, heterogenous, separation, texture), electrical (resistance, conductivity), or odorous.

8. Where the sensing process is activated, initiated or triggered in response to an event, action or another IA process.

9. The intelligent agent of claim 1, wherein the sensing circuitry is configured to sense more than one condition.

10. The intelligent agent of claim 1, wherein the communication circuitry comprises one or more of passive, active or wireless ad hoc power enabled wireless communication circuitry, or wired communication circuitry.

11. The intelligent agent of claim 1, wherein the power source is an onboard battery, energy harvester, or interface to an external power source, or a combination thereof.

12. The intelligent agent of claim 1, further comprising messaging circuitry.

13. The intelligent agent of claim 1, wherein progressive transformation of the sensed condition into determinations of the item's utility, includes the step of transforming the sensed condition into determinations of cumulative condition.

14. (canceled)

15. The intelligent agent of claim 1, wherein the progressive transformation of the sensed condition into a determination of the item's utility, includes logic equations, summing or averaging, weighted summing, multiplication, weighted multiplication, rule based or knowledge-based, fuzzy logic, data transformation, or cross terms or layered models.

16. The intelligent agent of claim 1, wherein one or more processes progressively evaluate determinations of the item's utility, and according to the results of the evaluations, (2) make decisions regarding actions to be performed.

17. The intelligent agent of claim 1, wherein progressive determinations of the item's utility are transformed into measures that are, along with their dependents, relevant to stakeholders in the item, its use or outcomes.

18. The intelligent agent of claim 17, wherein one or more processes progressively evaluate measures of the item's utility, and according to the results of the evaluations, (2) make decisions regarding actions to be performed.

19. The intelligent agent of claim 1, wherein the actions are from among: recording, storing, and generating proofs of and storing, data and information related to:
(1) events, conditions, stimuli
(2) determinations, measures, predictions, evaluations, and decisions, and their determinants, derivatives and dependents
(3) actions and transactions performed, and the results of them
(4) interactions with local and remote devices and systems
(5) communications and messaging
(6) changes to the control circuitry

20. The intelligent agent of claim 1, wherein the actions are (1) modifying, (2) updating, (3) adding to or deleting from, or (4) otherwise making changes to, the control circuitry, as allowed by the current instance of the control circuitry.

21. (canceled)

22. The intelligent agent of claim 1, wherein the actions are (1) activating, initiating or triggering one or more processes or (2) communicating or messaging, to or with, local or remote devices and systems, users or stakeholders.

23. The intelligent agent of claim 1, wherein in response to one or more events or conditions, determinations of cumulative condition or utility; evaluations, decisions or actions, or results of other processes, the control circuitry, adapts or modifies, activates, initiates or triggers processes to achieve predetermined targets or goals.
